(19) United States
(12) Patent Application Publication
(10) Pub. No.: US 2024/0068973 A1
(43) Pub. Date: Feb. 29, 2024

(54) SENSOR

(71) Applicant: KABUSHIKI KAISHA TOSHIBA, Tokyo (JP)

(72) Inventor: Yumi HAYASHI, Ayase Kanagawa (JP)

(73) Assignee: KABUSHIKI KAISHA TOSHIBA, Tokyo (JP)

(21) Appl. No.: 18/166,094

(22) Filed: Feb. 8, 2023

(30) Foreign Application Priority Data
Aug. 31, 2022 (JP) 2022-138493

(51) Int. Cl. G01N 27/22; G01N 27/407; G01N 27/416

(52) U.S. Cl. CPC G01N 27/226; G01N 27/221; G01N 27/227; G01N 27/4074; G01N 27/416; G01N 2027/222; G01N 33/005

(57) ABSTRACT

According to one embodiment, a sensor includes a structure. The structure includes a base layer, a first film, and a first layer. The first film includes at least one selected from a group consisting of silicon and aluminum, and oxygen. At least one of a volume of the first layer or an electrical resistance of the first layer is configured to change according to a detection target around the structure. The first film includes a first film region, a second film region, and a third film region. The first layer is between the base layer and the first film region in a first direction from the base layer to the first film region. The first layer is between the second film region and the third film region in a second direction crossing the first direction.

### CROSS-REFERENCE TO RELATED APPLICATIONS

This application is based upon and claims the benefit of priority from Japanese Patent Application No. 2022-138493, filed on Aug. 31, 2022; the entire contents of which are incorporated herein by reference.

### FIELD

Embodiments described herein relate generally to a sensor.

### BACKGROUND

For example, stable detection is desired for a sensor.

### BRIEF DESCRIPTION OF THE DRAWINGS

FIGS. 1A and 1B are schematic cross-sectional views illustrating a sensor according to a first embodiment;

FIG. 2 is a schematic cross-sectional view illustrating a sensor according to a second embodiment; and

FIG. 3 is a schematic cross-sectional view illustrating a sensor according to a third embodiment.

### DETAILED DESCRIPTION

According to one embodiment, a sensor includes a structure. The structure includes a base layer, a first film, and a first layer. The first film includes at least one selected from a group consisting of silicon and aluminum, and oxygen. At least one of a volume of the first layer or an electrical resistance of the first layer is configured to change according to a detection target around the structure. The first film includes a first film region, a second film region, and a third film region. The first layer is between the base layer and the first film region in a first direction from the base layer to the first film region. The first layer is between the second film region and the third film region in a second direction crossing the first direction.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously or illustrated in an antecedent drawing are marked with like reference numerals, and a detailed description is omitted as appropriate.

### First Embodiment

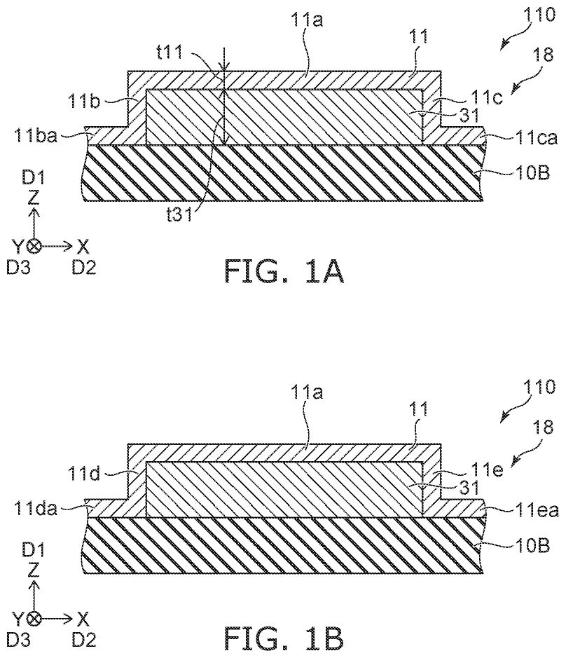

FIGS. 1A and 1B are schematic cross-sectional views illustrating a sensor according to a first embodiment.

As shown in FIGS. 1A and 1B, a sensor 110 according to the embodiment includes a structure 18. The structure 18 includes a base layer 10B, a first film 11 and a first layer 31.

The first film 11 includes at least one selected from the group consisting of silicon, aluminum, and oxygen. The first film 11 may include, for example, at least one selected from the group consisting of SiO₂ and Al₂O₃. The first film 11 may include SiAlO, for example.

At least one of a volume of the first layer 31 and an electrical resistance of the first layer 31 can change according to a detection target around the structure 18. For example, the detection target is gas. The detection target is, for example, hydrogen. The detection target may include at least one selected from the group consisting of hydrogen, hydrogen molecules, and hydrogen ions.

For example, the first layer 31 can incorporate the detection target (e.g., hydrogen). For example, the volume of the first layer 31 increases as the detection target is taken into the first layer 31. The change in the volume may cause the structure 18 to deform. For example, by detecting deformation, the concentration of the detection target can be detected.

The electrical resistivity of the first layer 31 changes (for example, increases) by taking the detection target into the first layer 31. This changes the electrical resistance of the first layer 31. By detecting the change in the electrical resistance, the concentration of the detection target can be detected.

As shown in FIG. 1A, the first film 11 includes a first film region 11a, a second film region 11b, and a third film region 11c. The first layer 31 is between the base layer 10B and the first film region 11a in a first direction D1 from the base layer 10B to the first film region 11a.

The first direction D1 is defined as a Z-axis direction. One direction perpendicular to the Z-axis direction is defined as an X-axis direction. A direction perpendicular to the Z-axis direction and the X-axis direction is defined as a Y-axis direction.

As shown in FIG. 1A, the first layer 31 is between the second film region 11b and the third film region 11c in a second direction D2. The second direction D2 crosses the first direction D1. In this example, the second direction D2 is the X-axis direction.

The first layer 31 is surrounded by the base layer 10B, the first film region 11a, the second film region 11b and the third film region 11c. The first layer 31 is covered with the base layer 10B and the first film 11. This suppresses deterioration of the first layer 31 due to external influences.

A detection target (for example, hydrogen) exists outside the structure 18. The detection target (for example, hydrogen) can pass through the first film 11 and reach the first layer 31. At least one of a change in volume or a change in electrical resistance occurs in the first layer 31 depending on the detection target.

A substance other than the detection target (for example, oxygen) exists outside the structure 18. When a substance other than the detection target (for example, oxygen) reaches the first layer 31, the first layer 31 may deteriorate. For example, when oxygen is taken into the first layer 31, the uptake of the detection target in the first layer 31 changes. For example, when oxygen is taken into the first layer 31, the volume change according to the detection target or the resistance change according to the detection target is not the intended state.

In the embodiment, the first layer 31 is provided between the base layer 10B and the first film 11. The first film 11 prevents substances other than the detection target (for example, oxygen) from reaching the first layer 31. This suppresses the adverse effects of substances other than the detection target (for example, oxygen). Stable detection is possible.

In the embodiment, for example, the first layer 31 includes Pd, Cu and Si. The first layer 31 may be a PdCuSi alloy. For example, the detection target includes hydrogen. When the first layer 31 includes PdCuSi, the detection target (hydrogen) is effectively taken into the first layer 31. At least one of the volume change or the resistance change effectively occurs.

The first layer 31 may further include at least one selected from the group consisting of Pt and Ti. These materials may, for example, function as catalysts. At least one of the volume change or the resistance change occurs more effectively.

At least a part of the first layer 31 is preferably amorphous. In the amorphous first layer 31, the detection target (for example, hydrogen) is taken in more effectively. For example, it is easy to obtain high sensitivity. For example, the signal obtained from structure 18 has a fast response to hydrogen. Hysteresis can be suppressed in hydrogen absorption and hydrogen release.

For example, when oxygen reaches the first layer 31, Si included in the first layer 31 may bond with oxygen. As a result, Pd and Cu included in the first layer 31 may combine to form crystals. The amorphous state of the first layer 31 may change to a crystalline state. This changes characteristic of at least one of the volume change or the resistance change.

The base layer 10B includes at least one selected from the group consisting of silicon, aluminum and titanium and at least one selected from the group consisting of oxygen and nitrogen. In one example, the base layer 10B includes at least one selected from the group consisting of silicon nitride and silicon oxide. The base layer 10B may include at least one selected from the group consisting of aluminum oxide and titanium nitride. The base layer 10B with stable characteristics is obtained.

As shown in FIG. 1A, the first film region 11a is continuous with the second film region 11b and the third film region 11c. For example, the second film region 11b and the third film region 11c may be in contact with the base layer 10B.

As shown in FIG. 1A, the first film 11 may further include a second extension region 11ba and a third extension region 11ca. The second extension region 11ba is continuous with the second film region 11b. The second extension region 11ba extends along the base layer 10B. The third extension region 11ca is continuous with the third film region 11c. The third extension region 11ca extends along the base layer 10B.

As shown in FIG. 1B, the first film 11 may include a fourth film region 11d and a fifth film region 11e. In the third direction D3, the first layer 31 is between the fourth film region 11d and the fifth film region 11e. The third direction D3 crosses a plane including the first direction D1 and the second direction D2. The third direction D3 may be, for example, the Y-axis direction.

For example, the first film region 11a is continuous with the fourth film region 11d and the fifth film region 11e. For example, the second film region 11b is continuous with the fourth film region 11d and the fifth film region 11e. For example, the third film region 11c is continuous with the fourth film region 11d and the fifth film region 11e. For example, the fourth film region 11d and the fifth film region 11e are in contact with the base layer 10B.

As shown in FIG. 1B, the first film 11 may further include a fourth extension region 11da and a fifth extension region ilea. The fourth extension region 11da is continuous with the fourth film region 11d. The fourth extension region 11da extends along the base layer 10B. The fifth extension region ilea is continuous with the fifth film region 11e. The fifth extension region ilea extends along the base layer 10B.

A thickness t31 (see FIG. 1A) of the first layer 31 along the first direction D1 is, for example, not less than 10 nm and not more than 10 μm.

A thickness t11 (see FIG. 1A) of the first film region 11a along the first direction D1 is, for example, not less than 1 nm and not more than 1000 nm.

### Second Embodiment

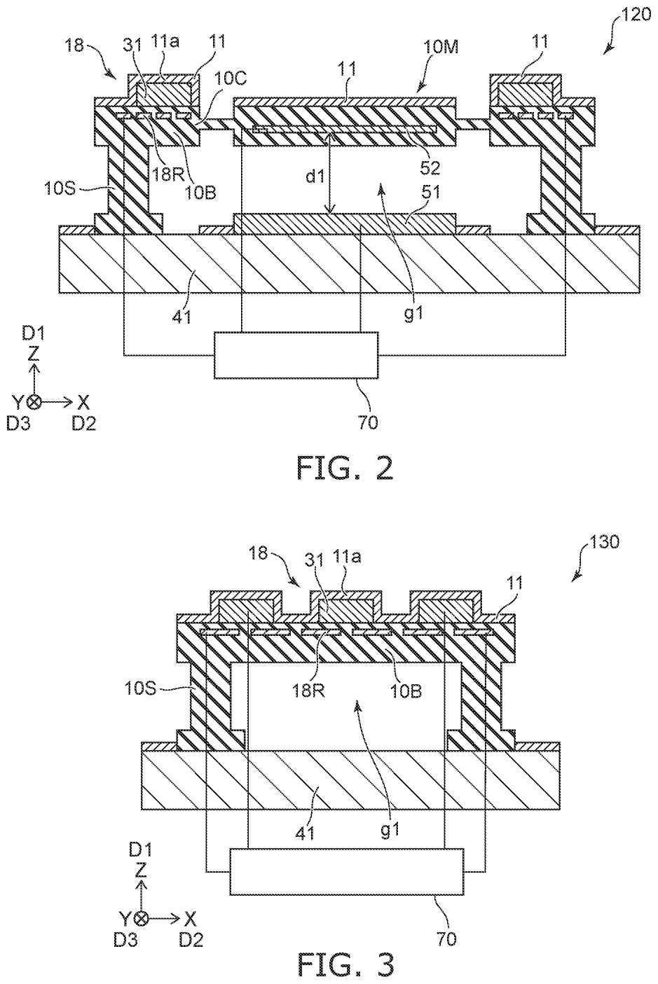

FIG. 2 is a schematic cross-sectional view illustrating a sensor according to a second embodiment.

As shown in FIG. 2, a sensor 120 according to the embodiment includes the structure 18, a base 41, a fixed electrode 51, a support part 10S and a movable electrode 52. The structure 18 has the configuration described with respect to the first embodiment.

The fixed electrode 51 is fixed to the base 41. The support part 10S is fixed to the base 41. The structure 18 is supported by the support part 10S. The movable electrode 52 is supported by the structure 18. A gap g1 is provided between the fixed electrode 51 and the movable electrode 52 and between the base 41 and the structure 18. The structure 18 functions as a connection part 10C. The connection part 10C supports a movable part 10M. The movable electrode 52 is included in the movable part 10M.

For example, the volume of the structure 18 changes according to the detection target. Thereby, the structure 18 is deformed. Thereby, a distance d1 between the fixed electrode 51 and the movable electrode 52 changes. A capacitance between the fixed electrode 51 and the movable electrode 52 changes due to the change in the distance d1. The capacitance can be changed according to the detection target. The detection target can be detected by detecting the capacitance.

As shown in FIG. 2, a circuit part 70 may be provided. The circuit part 70 may be included in sensor 120. The circuit part 70 may be provided separately from the sensor 120. The circuit part 70 can detect a value corresponding to the capacitance. The circuit part 70 outputs a signal corresponding to the capacitance.

As shown in FIG. 2, the structure 18 may further include a resistance member 18R. For example, at least a part of the resistance member 18R may overlap the first layer 31 in the first direction D1. For example, a current is supplied from the circuit part 70 to the resistance member 18R. As a result, the temperature of the resistance member 18R rises, and the temperature of the structure 18 rises. Accompanying this, the detection target uptake characteristics and release characteristics in the first layer 31 change. For example, detection with higher sensitivity becomes possible.

When the temperature of the first layer 31 repeatedly rises and falls, the deterioration of the first layer 31 is accelerated. By covering the first layer 31 with the first film 11, stable detection characteristics can be maintained even when the temperature is repeatedly increased and decreased.

### Third Embodiment

FIG. 3 is a schematic cross-sectional view illustrating a sensor according to a third embodiment.

As shown in FIG. 3, a sensor 130 according to the embodiment includes the structure 18, the base 41 and the support part 10S. The structure 18 has the configuration described with respect to the first embodiment.

The support part 10S is fixed to the base 41. The support part 10S supports the structure 18. The gap g1 is provided between the base 41 and the structure 18.

In the sensor 130, the electrical resistance of the first layer 31 included in the structure 18 changes according to the detection target. The detection target can be detected by detecting the electrical resistance.

The circuit part 70 may be provided. The circuit part 70 can detect a value corresponding to the electrical resistance of the first layer 31. A signal corresponding to the electrical resistance of the first layer 31 is output from the circuit part 70.

As shown in FIG. 3, the structure 18 may further include the resistance member 18R. For example, at least a part of the resistance member 18R may overlap the first layer 31 in the first direction D1.

For example, the temperature of resistance member 18R increases, and the temperature of structure 18 increases. Accompanying this, the detection target uptake characteristics and release characteristics in the first layer 31 change. For example, detection with higher sensitivity becomes possible. When the temperature of the first layer 31 repeatedly rises and falls, the deterioration of the first layer 31 is accelerated. By covering the first layer 31 with the first film 11, stable detection characteristics can be maintained even when the temperature is repeatedly increased and decreased.

Embodiments may include the following configurations (for example, technical proposals).

### Configuration 1

A sensor comprising:
a structure including
   a base layer,
   a first film, and
   a first layer,
the first film including at least one selected from a group consisting of silicon and aluminum, and oxygen,
at least one of a volume of the first layer or an electrical resistance of the first layer being configured to change according to a detection target around the structure,
the first film including a first film region, a second film region, and a third film region,
the first layer being between the base layer and the first film region in a first direction from the base layer to the first film region, and
the first layer being between the second film region and the third film region in a second direction crossing the first direction.

### Configuration 2

The sensor according to Configuration 1, wherein
the film region is continuous with the second film region and the third film region.

### Configuration 3

The sensor according to Configuration 1 or 2, wherein
the second film region and the third film region are in contact with the base layer.

### Configuration 4

The sensor according to any one of Configurations 1 to 3, wherein
the first film further includes a second extension region and a third extension region,
the second extension region is continuous with the second film region,
the second extension region extends along the base layer,
the third extension region is continuous with the third film region, and
the third extension region extends along the base layer.

### Configuration 5

The sensor according to any one of Configurations 1 to 4, wherein
the first film includes a fourth film region and a fifth film region, and
the first layer is between the fourth film region and the fifth film region in a third direction crossing a plane including the first direction and the second direction.

### Configuration 6

The sensor according to Configuration 5, wherein
the first film region is continuous with the fourth film region and the fifth film region.

### Configuration 7

The sensor according to Configuration 5 or 6, wherein
the fourth film region and the fifth film region are in contact with the base layer.

### Configuration 8

The sensor according to any one of Configurations 5 to 7, wherein
the first film further includes a fourth extension region and a fifth extension region,
the fourth extension region is continuous with the fourth film region,
the fourth extension region extends along the base layer,
the fifth extension region is continuous with the fifth film region, and
the fifth extension region extends along the base layer.

### Configuration 9

The sensor according to any one of Configurations 1 to 8, wherein
the first layer includes Pd, Cu, and Si.

### Configuration 10

The sensor according to Configuration 9, wherein the first layer further includes at least one selected from a
group consisting of Pt and Ti.

### Configuration 11

The sensor according to Configuration 9 or 10, wherein
at least a part of the first layer is amorphous.

### Configuration 12

The sensor according to any one of Configurations 9 to 11, wherein
the detection target includes hydrogen.

### Configuration 13

The sensor according to any one of Configurations 1 to 12, further comprising:
a base;
a fixed electrode fixed to the base;
a support part fixed to the base; and
a movable electrode,
the structure being supported by the support part,
the movable electrode being supported by the structure, and
a gap being provided between the fixed electrode and the movable electrode, and between the base and the structure.

### Configuration 14

The sensor according to Configuration 13, wherein
the structure further includes a resistance member, and
at least a part of the resistance member overlaps the first layer in the first direction.

### Configuration 15

The sensor according to Configuration 13 or 14, wherein
capacitance between the fixed electrode and the movable electrode is configured to change according to the detection target.

### Configuration 16

The sensor according to Configuration 15, further comprising:
a circuit part configured to detect a value corresponding to the capacitance.

### Configuration 17

The sensor according to any one of Configurations 1 to 12, further comprising:
a base; and
a support part fixed to the base,
the support part supporting the structure, and
a gap being provided between the base and the structure.

### Configuration 18

The sensor according to Configuration 17, wherein
the structure further includes a resistance member, and
at least a part of the resistance member overlaps the first layer in the first direction.

### Configuration 19

The sensor according to Configuration 17 or 18, further comprising:
a circuit part configured to detect a value corresponding to the electrical resistance.

According to the embodiments, it is possible to provide a sensor capable of stable detection.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in sensors such as structures, layers, films, circuit parts, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors practicable by an appropriate design modification by one skilled in the art based on the sensors described above as embodiments of the invention also are within the scope of the invention to the extent that the spirit of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor comprising:
a structure including
a base layer,
a first film, and
a first layer,
the first film including at least one selected from a group consisting of silicon and aluminum, and oxygen,
at least one of a volume of the first layer or an electrical resistance of the first layer being configured to change according to a detection target around the structure,
the first film including a first film region, a second film region, and a third film region,
the first layer being between the base layer and the first film region in a first direction from the base layer to the first film region, and
the first layer being between the second film region and the third film region in a second direction crossing the first direction.

2. The sensor according to claim 1, wherein
the film region is continuous with the second film region and the third film region.

3. The sensor according to claim 1, wherein
the second film region and the third film region are in contact with the base layer.

4. The sensor according to claim 1, wherein
the first film further includes a second extension region and a third extension region,
the second extension region is continuous with the second film region,
the second extension region extends along the base layer,
the third extension region is continuous with the third film region, and
the third extension region extends along the base layer.

5. The sensor according to claim 1, wherein
the first film includes a fourth film region and a fifth film region, and
the first layer is between the fourth film region and the fifth film region in a third direction crossing a plane including the first direction and the second direction.

6. The sensor according to claim 5, wherein
the first film region is continuous with the fourth film region and the fifth film region.

7. The sensor according to claim 5, wherein
the fourth film region and the fifth film region are in contact with the base layer.

8. The sensor according to claim 5, wherein
the first film further includes a fourth extension region and a fifth extension region,
the fourth extension region is continuous with the fourth film region,
the fourth extension region extends along the base layer,
the fifth extension region is continuous with the fifth film region, and
the fifth extension region extends along the base layer.

9. The sensor according to claim 1, wherein
the first layer includes Pd, Cu, and Si.

10. The sensor according to claim 9, wherein
the first layer further includes at least one selected from a group consisting of Pt and Ti.

11. The sensor according to claim 9, wherein
at least a part of the first layer is amorphous.

12. The sensor according to claim 9, wherein
the detection target includes hydrogen.

13. The sensor according to claim 1, further comprising:
a base;
a fixed electrode fixed to the base;
a support part fixed to the base; and
a movable electrode,
the structure being supported by the support part,
the movable electrode being supported by the structure, and
a gap being provided between the fixed electrode and the movable electrode, and between the base and the structure.

14. The sensor according to claim 13, wherein
the structure further includes a resistance member, and
at least a part of the resistance member overlaps the first layer in the first direction.

15. The sensor according to claim 13, wherein
capacitance between the fixed electrode and the movable electrode is configured to change according to the detection target.

16. The sensor according to claim 15, further comprising:
a circuit part configured to detect a value corresponding to the capacitance.

17. The sensor according to claim 1, further comprising:
a base; and
a support part fixed to the base,
the support part supporting the structure, and
a gap being provided between the base and the structure.

18. The sensor according to claim 17, wherein
the structure further includes a resistance member, and
at least a part of the resistance member overlaps the first layer in the first direction.

19. The sensor according to claim 17, further comprising:
a circuit part configured to detect a value corresponding to the electrical resistance.
